(19) United States
(12) Patent Application Publication
(10) Pub. No.: US 2022/0347247 A1
(43) Pub. Date: Nov. 3, 2022

(54) USE OF CANNABINOIDS IN THE TREATMENT OF INFLAMMATION AND AGING IN THE SKIN

(71) Applicant: Demetrix, Inc., Berkeley, CA (US)

(72) Inventors: Cynthia W. Bryant, Raleigh, NC (US); Alison Watta, Durham, NC (US)

(21) Appl. No.: 17/661,274

(22) Filed: Apr. 28, 2022

Related U.S. Application Data
(60) Provisional application No. 63/181,041, filed on Apr. 28, 2021.

(51) Int. Cl. A61K 36/185; A61K 31/05; A61K 31/352

(52) U.S. Cl. CPC A61K 36/185; A61K 31/05; A61K 31/352

(57) ABSTRACT

The present disclosure relates to compositions and methods for reducing, preventing, and/or treating skin conditions comprising the administration of one or more cannabidiols. Specifically, the present disclosure relates to compositions comprising CBG or CBGVA for the reduction of aging.

### CROSS-REFERENCE TO RELATED APPLICATIONS

The present application claims the priority benefit of U.S. Provisional Patent App. Ser. No. 63/181,041, filed Apr. 28, 2021, which is hereby incorporated herein by reference.

### TECHNICAL FIELD

The present disclosure relates generally to the use of cannabinoids in in the treatment or prevention of inflammation and aging in the skin. Specifically, CBG, CBGVA or combinations thereof are useful in reducing inflammation and aging in the skin.

### BACKGROUND

Aging of human skin is due to both intrinsic (chronological) factors, and extrinsic (external factors). Inflammation plays a role in both the intrinsic and the extrinsic factors of aging. Specifically, the extrinsic causes of inflammation are due to factors such as sun exposure, pollutants, smoking and stress. Inflammation in the skin can lead to reduced collagen production and increased activity of matrix metalloproteinases. Interleukin 8 (IL-8) is a proinflammatory cytokine known to play a role in inflammatory skin diseases such as psoriasis. Reduction in IL-8 has been shown to correlate with improvement in symptoms for patients with Atopic Dermatitis (Murata, Et al.)

Additionally, prostaglandin E2 (PGE₂) levels increase when skin is exposed to ultraviolet radiation (UVR). It has also been shown that PGE₂ levels in the skin gradually increase with age which causes low level, chronic inflammation that continually damages the dermal matrix (Fuller et al., Cosmetics 2019, 6, 6). PGE₂ has been shown to negatively regulate an atopic dermatitis skin model in mice by suppressing thymic stromal lymphopoietin (TSLP) expression (Eyerich et al., J Allergy Clin Immunol 2019; 144:1175-6). PGE-2 exerts its effects on target cells by binding to and activating one or more of four PGE-2 receptors, known as EP1, EP2, EP3, and EP4 (Fuller).

Thus, an active ingredient in a composition that regulates and/or inhibits PGE₂ expression may be successfully used as an inflammatory ingredient in skincare, personal care, and prescription products to treat, reduce, and prevent both intrinsic and extrinsic inflammation leading to a reduction in skin aging. The present disclosure directly addresses this significant unmet need.

### SUMMARY

The present disclosure is directed to a composition for use as a skincare product to reduce, treat, prevent, or ameliorate a skin condition, wherein the composition comprises cannabigerol (CBG), cannabigerovarinic acid (CBGVA) or a combination thereof.

In some embodiments, the composition comprises effective amounts of CBG, CBGVA, or a combination thereof in an amount effective in reducing, treating, preventing, or ameliorating the skin condition and/or reducing, treating, preventing, or ameliorating inflammation.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBG.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBGVA.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBG and CBGVA, collectively.

In some embodiments, the composition comprises less than 5%, less than 4%, less than 3%, less than 2%, less than 1%, less than 0.5%, or less than 0.1% cannabinoids other than CBG and/or CBGVA.

In some embodiments, the composition is a purified extract of a cannabis plant.

In some embodiments, the composition is formulated for topical application.

In some embodiments, the composition is a lotion, a cream, or an ointment.

In some embodiments, the composition is formulated for oral administration.

In some embodiments, the composition is a pill, an oil, a syrup or a tablet.

In some embodiments, the skin condition is an inflammatory condition.

In some embodiments, the inflammatory condition is an acute inflammatory condition.

In some embodiments, the inflammatory condition is a chronic inflammatory condition.

In some embodiments, the inflammatory condition is associated with increased Prostaglandin E2 (PGE2) expression.

In some embodiments, the skin condition is atopic dermatitis.

In some embodiments, the skin condition is a sign or symptom of ageing.

In some embodiments, the sign or symptom of ageing is wrinkling of the skin.

In some embodiments, the condition is the dermatological symptom of an immunological disorder.

In some embodiments, the administration of the composition leads to a decrease in PGE2 production in the skin.

In some embodiments, the composition comprises one or more additional active ingredient.

In one aspect, the present disclosure is directed to a method of producing the compositions described herein, comprising mixing the cannabigerol (CBG), cannabigerovarinic acid (CBGVA) or a combination thereof, and a suitable carrier.

In one aspect, the present disclosure is directed to a method of reducing, treating, preventing, or ameliorating the skin condition and/or reducing, treating, preventing, or ameliorating inflammation in subject in need thereof, comprising administering an effective amount of the composition of the present disclosure to the subject.

In one aspect, the present disclosure is directed to a method of reducing, treating, preventing, or ameliorating inflammation in subject in need thereof, comprising administering an effective amount of the composition of the present disclosure to the subject.

In one aspect, the present disclosure is directed to a method of reducing, treating, preventing, or ameliorating the skin condition and/or reducing, treating, preventing, or ameliorating inflammation in subject in need thereof, comprising administering an effective amount of the composition of the present disclosure to the subject.

In one aspect, the present disclosure is directed to a method of decreasing release of IL-8 by keratinocytes, comprising contacting the keratinocytes with an effective amount of the composition of the present disclosure.

In one aspect, the present disclosure is directed to a method of decreasing release of PGE2 by keratinocytes, comprising contacting the keratinocytes with an effective amount of the composition of the present disclosure.

In some embodiments, the keratinocytes are human epidermal keratinocytes.

### DETAILED DESCRIPTION

### Definitions

As used throughout, ranges are used as shorthand for describing each and every value that it is within the range. Any value within the range can be selected as the terminus of the range.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about”, when applied to the value for a parameter of a composition or method of this invention, indicates that the calculation or the measurement of the value allows some slight imprecision without having a substantial effect on the chemical or physical attributes of the composition or the method. If, for some reason, the imprecision provided by “about” is not otherwise understood in the art with this ordinary meaning, then “about” as used herein indicates a possible variation of up to 5% in the value.

As referred to herein, compositional percentages are by weight of the total composition, unless otherwise specified.

As used herein, “ppm” (parts per million) refers to ppm by weight, unless otherwise indicated.

As referred to herein, all ratios refer to weight ratios, unless otherwise indicated.

As used herein, the term “treating” may refer to, for example, an improvement of one of more symptoms of a condition and/or a delay in disease progression. As used herein, the term “preventing” may refer to, for example, a delay in disease onset compared to, e.g., a population average.

The term “one or more cannabinoid” generally refers to CBG either alone or in combination with one or more as discussed herein. The term “one or more cannabinoid” generally refers to CBGVA either alone or in combination with one or more as discussed herein.

As used herein, “treat” and variations thereof as used herein refers to cure, ameliorate, alleviate, inhibit, prevent, reduce the likelihood of, or reduce the severity of, a disease or condition, or of at least some of the symptoms or effects thereof. To treat a tissue of the mouth, the tissue is contacted with the composition effective at treating the condition. In some embodiments, oral inflammation may be contacted with an oral care composition comprising CBG, CBGVA, or a combination thereof.

### Compositions

In one aspect, provided herein are compositions for use as a skincare product, wherein the composition comprises an amount of cannabigerol (CBG) and/or cannabigerovarinic acid (CBGVA) effective in reducing, treating, preventing, or ameliorating a skin condition.

The exact amount of CBG or CBGVA which is effective to reduce, treat, prevent or ameliorate a skin condition may vary depending on the condition and the formulation of the composition. In some embodiments, a composition comprises both CBG and CBGVA. The ratio of CBG to CBGVA in the composition may also vary. In some embodiments, a composition comprises CBG but is substantially free of CBGVA. In some embodiments, a composition comprises CBGVA but is substantially free of CBG.

As described herein, some cannabinoids are able to inhibit Prostaglandin E2 (PGE₂) in Normal Human Epidermal Keratinocyte (NHEK) cells (see Example 1). Without wishing to be bound by any particular theory or mechanism, as an inhibitor of PGE₂, select cannabinoids can therefore help prevent both acute and chronic skin inflammation, including inflammation due to intrinsic and extrinsic aging of the skin. However, not all cannabinoids have anti-PGE₂ activity in all tissue types and, unpredictably, some cannabinoids actually lead to an increase PGE₂ and thus would be predisposed to increase inflammation. Thus, cannabidiol (CBD) and cannabigerolic acid (CBGA) were both shown to stimulate PGE₂ expression and would thus be expected to increase inflammation.

Therefore, without wishing to be bound by any particular theory or mechanism, by incorporating only those cannabinoids that have a reductive effect on PGE₂ production, the present disclosure offers compositions and methods of using the compositions that provide specificity anti-aging and/or anti-inflammatory properties.

In some embodiments, a composition provided herein is substantially free of cannabinoids other than CBG and/or CBGVA. For example, the composition may comprise less than 5%, less than 4%, less than 3%, less than 2%, less than 1%, less than 0.5%, or less than 0.1% cannabinoids other than CBG and/or CBGVA. “Cannabinoids” include, without limitation, CBG, CBGVA, cannabidiol (CBD), cannabigerolic acid (CBGA), and cannabidiolic acid (CBDA). Thus, in one embodiment, the composition described herein is substantially free of CBD. In one embodiment, the composition described herein is substantially free of CBGA. In one embodiment, the composition described herein is substantially free of CBDA. In one embodiment, the composition described herein is substantially free of CBD and CBGA. In one embodiment, the composition described herein is substantially free of CBD and CBDA. In one embodiment, the composition described herein is substantially free of CBGA and CBDA. In one embodiment, the composition described herein is substantially free of CBGA, CBDA, and CBD.

Prior to the present discovery, it was not appreciated which specific cannabinoid compounds could act to treat skin inflammation, skin aging, and associated conditions. By selecting the cannabinoid compounds with the greatest effectiveness against skin inflammation and skin aging, the present disclosure provides improved methods of improving skin health.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to treat skin conditions with specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally derived cannabinoid compounds offer benefits in regard to the treatment of skin inflammation and skin aging not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of treating skin inflammation and skin aging that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally derived cannabinoid compounds are sufficiently pure and/or if any containments are sufficiently well understood.

In some embodiments, the composition of the present disclosure comprises CBG at a concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises CBG at a concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBG.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of CBG.

In some embodiments, the composition of the present disclosure comprises CBGVA at a concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises CBGVA at a concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBGVA.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of CBGVA.

In some embodiments, the composition of the present disclosure comprises CBG and CBGVA in a combined concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises CBG and CBGVA in a combined concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of both CBG and CBGVA, collectively.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of both CBG and CBGVA, collectively.

In some embodiments, the composition of the present disclosure comprises CBG, CBGVA, or a combination thereof at a concentration of between at least about 0.1 μM and 1 mM. In some embodiments, the composition of the present disclosure comprises CBG, CBGVA, or a combination thereof at a concentration of between at least about 0.5 μM and 1 mM, between at least about 1 μM and 1 mM, between at least about 2 μM and 1 mM, between at least about 5 μM and 1 mM, between at least about 10 μM and 1 mM, between at least about 15 μM and 1 mM, between at least about 20 μM and 1 mM, between at least about 25 μM and 1 mM, between at least about 50 μM and 1 mM, between at least about 100 μM and 1 mM, between at least about 150 μM and 1 mM, between at least about 200 μM and 1 mM, between at least about 250 μM and 1 mM, between at least about 300 μM and 1 mM, between at least about 350 μM and 1 mM, between at least about 400 μM and 1 mM, between at least about 450 μM and 1 mM, or between at least about 500 μM and 1 mM.

In some embodiments, the composition of the present disclosure comprises CBG, CBGVA, or a combination thereof at a concentration of between at least about 0.1 μM and 500 μM. In some embodiments, the composition of the present disclosure comprises CBG, CBGVA, or a combination thereof at a concentration of between at least about 0.5 μM and 500 μM, between at least about 1 μM and 500 μM, between at least about 2 μM and 500 μM, between at least about 5 μM and 500 μM, between at least about 10 μM and 500 μM, between at least about 15 μM and 500 μM, between at least about 20 μM and 500 μM, between at least about 25 μM and 500 μM, between at least about 50 μM and 500 μM, between at least about 100 μM and 500 μM, between at least about 150 μM and 500 μM, between at least about 200 μM and 500 μM, between at least about 250 μM and 500 μM, between at least about 300 μM and 500 μM, between at least about 350 μM and 500 μM, between at least about 400 μM and 500 μM, or between at least about 450 μM and 500 μM.

In some embodiments, the composition of the present disclosure comprises CBG, CBGVA, or a combination thereof at a concentration of between at least about 0.1 μM and 250 μM. In some embodiments, the composition of the present disclosure comprises CBG, CBGVA, or a combination thereof at a concentration of between at least about 0.5 μM and 250 μM, between at least about 1 μM and 250 μM, between at least about 2 μM and 250 μM, between at least about 5 μM and 250 μM, between at least about 10 μM and 250 μM, between at least about 15 μM and 250 μM, between at least about 20 μM and 250 μM, between at least about 25 μM and 250 μM, between at least about 50 μM and 250 μM, between at least about 100 μM and 250 μM, between at least about 150 μM and 250 μM, or between at least about 200 μM and 250 μM.

In some embodiments, the composition of the present disclosure comprises CBG, CBGVA, or a combination thereof at a concentration of between at least about 0.1 μM and 100 μM. In some embodiments, the composition of the present disclosure comprises CBG, CBGVA, or a combination thereof at a concentration of between at least about 0.5 μM and 100 μM, between at least about 1 μM and 100 μM, between at least about 2 μM and 100 μM, between at least about 5 μM and 100 μM, between at least about 10 μM and 100 μM, between at least about 15 μM and 100 μM, between at least about 20 μM and 100 μM, between at least about 25 μM and 100 μM, or between at least about 50 μM and 100 μM.

In some embodiments, the composition of the present disclosure comprises CBG, CBGVA, or a combination thereof at a concentration of between at least about 0.1 μM and 75 μM. In some embodiments, the composition of the present disclosure comprises CBG, CBGVA, or a combination thereof at a concentration of between at least about 0.5 μM and 75 μM, between at least about 1 μM and 75 μM, between at least about 2 μM and 75 μM, between at least about 5 μM and 75 μM, between at least about 10 μM and 75 μM, between at least about 15 μM and 75 μM, between at least about 20 μM and 100 μM, between at least about 25 μM and 75 μM, or between at least about 50 μM and 75 μM.

In some embodiments, the composition of the present disclosure comprises CBG, CBGVA, or a combination thereof at a concentration of between at least about 0.1 μM and 50 μM. In some embodiments, the composition of the present disclosure comprises CBG, CBGVA, or a combination thereof at a concentration of between at least about 0.5 μM and 50 μM, between at least about 1 μM and 50 μM, between at least about 2 μM and 50 μM, between at least about 5 μM and 50 μM, between at least about 10 μM and 50 μM, between at least about 15 μM and 50 μM, between at least about 20 μM and 50 μM, or between at least about 25 μM and 50 μM.

In some embodiments, the composition of the present disclosure comprises CBG, CBGVA, or a combination thereof at a concentration of between at least about 0.1 μM and 25 μM. In some embodiments, the composition of the present disclosure comprises CBG, CBGVA, or a combination thereof at a concentration of between at least about 0.5 μM and 25 μM, between at least about 1 μM and 25 μM, between at least about 2 μM and 25 μM, between at least about 5 μM and 25 μM, between at least about 10 μM and 25 μM, between at least about 15 μM and 25 μM, or between at least about 20 μM and 25 μM.

In some embodiments, the composition of the present disclosure comprises CBG, CBGVA, or a combination thereof at a concentration of between at least about 0.1 μM and 20 μM. In some embodiments, the composition of the present disclosure comprises CBG, CBGVA, or a combination thereof at a concentration of between at least about 0.5 μM and 20 μM, between at least about 1 μM and 20 μM, between at least about 2 μM and 20 μM, between at least about 5 μM and 20 μM, between at least about 10 μM and 20 μM, or between at least about 15 μM and 20 μM.

In some embodiments, the composition of the present disclosure comprises CBG, CBGVA, or a combination thereof at a concentration of between at least about 0.1 μM and 15 μM. In some embodiments, the composition of the present disclosure comprises CBG, CBGVA, or a combination thereof at a concentration of between at least about 0.5 μM and 15 μM, between at least about 1 μM and 15 μM, between at least about 2 μM and 15 μM, between at least about 5 μM and 15 μM, between at least about 10 μM and 15 μM.

In some embodiments, the composition of the present disclosure comprises CBG, CBGVA, or a combination thereof at a concentration of between at least about 0.1 μM and 10 μM. In some embodiments, the composition of the present disclosure comprises CBG, CBGVA, or a combination thereof at a concentration of between at least about 0.5 μM and 10 μM, between at least about 1 μM and 10 μM, between at least about 2 μM and 10 μM, between at least about 5 μM and 10 μM.

In some embodiments, the composition of the present disclosure comprises CBG, CBGVA, or a combination thereof at a concentration of between at least about 0.1 μM and 5 μM. In some embodiments, the composition of the present disclosure comprises CBG, CBGVA, or a combination thereof at a concentration of between at least about 0.5 μM and 5 μM, between at least about 1 μM and 5 μM, between at least about 2 μM and 5 μM.

In some embodiments, the composition of the present disclosure comprises CBG, CBGVA, or a combination thereof at a concentration of between at least about 0.1 μM and 2 μM. In some embodiments, the composition of the present disclosure comprises CBG, CBGVA, or a combination thereof at a concentration of between at least about 0.5 μM and 2 μM, between at least about 1 μM and 2 μM.

In some embodiments, the composition of the present disclosure comprises CBG, CBGVA, or a combination thereof at a concentration of between at least about 0.1 μM and 1 μM. In some embodiments, the composition of the present disclosure comprises CBG, CBGVA, or a combination thereof at a concentration of between at least about 0.5 μM and 1 μM.

In some embodiments, the composition of the present disclosure comprises CBG, CBGVA, or a combination thereof at a concentration of between at least about 0.1 μM and 0.5 μM.

In some embodiments, the composition of the present disclosure comprises a combination of CBG and CBVGA.

In some embodiments, the combination is a 9:1 CBG:CBGVA ratio.

In some embodiments, the combination is a 8:1 CBG:CBGVA ratio.

In some embodiments, the combination is a 7:1 CBG:CBGVA ratio.

In some embodiments, the combination is a 6:1 CBG:CBGVA ratio.

In some embodiments, the combination is a 5:1 CBG:CBGVA ratio.

In some embodiments, the combination is a 4:1 CBG:CBGVA ratio.

In some embodiments, the combination is a 3:1 CBG:CBGVA ratio.

In some embodiments, the combination is a 2:1 CBG:CBGVA ratio.

In some embodiments, the combination is a 1:1 CBG:CBGVA ratio.

In some embodiments, the combination is a 1:2 CBG:CBGVA ratio.

In some embodiments, the combination is a 1:3 CBG:CBGVA ratio.

In some embodiments, the combination is a 1:4 CBG:CBGVA ratio.

In some embodiments, the combination is a 1:5 CBG:CBGVA ratio.

In some embodiments, the combination is a 1:6 CBG:CBGVA ratio.

In some embodiments, the combination is a 1:7 CBG:CBGVA ratio.

In some embodiments, the combination is a 1:8 CBG:CBGVA ratio.

In some embodiments, the combination is a 1:9 CBG:CBGVA ratio.

In some embodiments, the composition is a natural product, e.g., an extract of a cannabis plant. In some embodiments, the composition is a concentrated extract of a plant belonging to the cannabis genus. In some embodiments, the composition is a synthetic product. In some embodiments, the cannabinoids discussed herein are produced via fermentation.

### Formulations

A composition provided herein may be formulated for any suitable route of administration. In some embodiments, the composition is formulated for oral administration, e.g., the composition is pill, an oil, a syrup or a tablet. In some embodiments, the composition is formulated for topical administration, e.g., the composition is a lotion, a cream, or an ointment.

The composition described herein also comprises a pharmaceutically acceptable carrier. The pharmaceutically acceptable carrier may be a carrier generally regarded as safe (GRAS). Conventional procedures for the selection and preparation of suitable GRAS formulations are described in, for example, “Pharmaceuticals—The Science of Dosage Form Designs,” M. E. Aulton, Churchill Livingstone, 1988, which is hereby incorporated by reference in its entirety.

The term “carrier,” as used in this disclosure, may encompass carriers, excipients, and diluents and may mean a material, composition or vehicle, such as a liquid or solid filler, diluent, excipient, solvent or encapsulating material, involved in carrying or transporting an agent (e.g., CBG or CBGVA) from one organ, or portion of the body, to another organ, or portion of the body of a subject. Carriers should be selected on the basis of compatibility and the release profile properties of the desired dosage form. Carriers may also be selected because they are GRAS. Exemplary carrier materials may include, e.g., binders, suspending agents, disintegration agents, filling agents, surfactants, solubilizers, stabilizers, lubricants, wetting agents, diluents, spray-dried dispersions, and the like. See, e.g., Hoover, John E., Remington's Pharmaceutical Sciences, Mack Publishing Co., Easton, Pa. 1975.

The present disclosure provides a process for the preparation of a composition described herein which comprises mixing CBG and/or CBGVA with an acceptable carrier. Compositions described herein can also be prepared according to conventional mixing, granulating, or coating methods.

Some examples of materials that can serve as acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants and can also be present in the compositions provided herein, according to the judgment of the formulator.

Depending on the intended mode of administration, the composition described herein can be in solid, semi-solid or liquid dosage form, such as, for example, injectables, tablets, suppositories, pills, softgels, time-release capsules, elixirs, tinctures, oils, extracts, creams, lotions, emulsions, syrups, powders, liquids, suspensions, or the like, sometimes in unit dosages and consistent with conventional practices. These modes may include systemic or local administration such as oral, transdermal, or topical (as by powders, ointments, or drops) administration modes.

Solid dosage forms of a composition described herein for oral administration may include capsules, softgels, tablets, pills, powders, crystals, and granules. In such solid dosage forms, one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) may be mixed with at least one inert, acceptable or pharmaceutically acceptable carriers such as a diluent, fillers or extenders, binders, humectants, disintegrating agents, solution retarding agents, wetting agents, lubricants, an emulsifier or dispersing agent, or buffering agents.

Solid formulations of a similar type may also be employed as fillers in soft and hard-filled gelatin capsules using such excipients as lactose or milk sugar as well as high molecular weight polyethylene glycols and the like.

The solid dosage forms of tablets, dragees, softgels, capsules, pills, crystals, and granules can be prepared with coatings and shells such as enteric coatings, release controlling coatings and other coatings well known in the art. In such solid dosage forms, CBG and/or CBGVA may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, softgels, tablets and pills, the dosage forms may also comprise buffering agents. They may optionally contain opacifying agents and can also be of a formulation that delay the release of the CBG and/or CBGVA, only, or preferentially, in a some part of the intestinal tract, optionally. Examples of embedding compositions that can be used may include polymeric substances and waxes.

Liquid dosage forms of a composition described herein for oral administration may include emulsions, microemulsions, solutions, suspensions, syrups, tinctures, oils, extracts, and elixirs. In addition to CBG and/or CBGVA, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral formulations can also include adjuvants such as wetting agents, emulsifying and suspending agents, sweetening, flavoring, and perfuming agents.

Dosage forms for topical or transdermal administration of a composition described herein include ointments, pastes, creams, lotions, gels, powders, solutions, sprays, inhalants, oils, or patches. The CBG and/or CBGVA are admixed under sterile conditions with an acceptable or pharmaceutically acceptable carrier and any needed preservatives or buffers as may be required. Ophthalmic formulations, ear drops, and the like are also contemplated as being within the scope of this disclosure.

The ointments, pastes, creams, lotions, gels, solutions, inhalants, or oils may contain, in addition to CBG and/or CBGVA, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

A composition provided herein may also be formulated for use as topical powders and sprays that can contain, in addition to one or more cannabinoid or cannabinoid derivative preparations, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of CBG and/or CBGVA to the body. Such dosage forms can be made by dissolving or dispensing the CBG and/or CBGVA in the proper medium. Absorption enhancers can also be used to increase the flux of CBG and/or CBGVA across the skin. The rate can be controlled by either providing a rate controlling membrane or by dispersing the CBG and/or CBGVA in a polymer matrix or gel.

In some embodiments, a composition described herein is an edible formulation comprising CBG and/or CBGVA. “Edible formulation” may refer to a composition suitable for consumption, typically via the oral cavity (although consumption may occur via non-oral means such as inhalation). Edible formulations may be present in any form including, but not limited to, liquids, solids, semi-solids, tablets, lozenges, powders, gels, gums, pastes, slurries, syrups, aerosols, and sprays. As used herein, edible formulations may include food products, pharmaceutical formulations, and consumer products. Edible formulations may also refer to, for example, dietary and nutritional supplements. As used herein, edible formulations may also include formulations that are placed within the oral cavity but not swallowed.

“Food product” may refer to any formulations comprising one or more processed foodstuff. Food products include, but are not limited to, confectionaries, bakery products, ice creams, dairy products, cheeses, sweet and savory snacks, snack foods, beverages (including, but not limited to, hot and cold beverages, beverage mixes, concentrates, juices, carbonated beverages, non-carbonated beverages, alcoholic beverages, non-alcoholic beverages, soft drinks, sports drinks, isotonic drinks, coffees, teas, bottled waters, and beverages prepared from botanicals and botanical extracts), snack bars, meal replacement products, ready meals (including, but not limited to canned meals, preserved meals, frozen meals, dried meals, chilled meals, dinner mixes, and prepared salads), soups, broth, prepared foods (including, but not limited to, dried, canned, or jarred sauces and soups), canned foods, frozen foods, dried foods, chilled foods, oils and fats, sauces, jellies, jams, preserves, honey, puddings, recipe mixes, syrups, icings, fillings, infused foods, and condiments.

“Foodstuff” may refer to an unprocessed ingredient or a basic nutrient or flavor containing element used to prepare a food product. Non-limiting examples of foodstuffs include: fruits, vegetables, meats, fishes, grains, milks, eggs, tubers, sugars, sweeteners, oils, herbs, snacks, sauces, spices and salts.

In some embodiments, a composition described herein may be a consumer product comprising CBG and/or CBGVA. “Consumer products” may refer to health, beauty, and general wellness products for the personal use and/or consumption by a subject. Consumer products may be present in any form including, but not limited to, liquids, solids, semi-solids, tablets, capsules, lozenges, strips, powders, gels, gums, pastes, slurries, syrups, aerosols and sprays. Non-limiting examples of consumer products include nutraceuticals, nutritional supplements, cosmetics, sunscreens, lotions, creams, wipes, lipsticks, lip balms, soaps, shampoos, gums, dissolvable films, adhesives (e.g., dental adhesives), toothpastes, breath fresheners, mouthwashes, and other dentifrices.

In some embodiments wherein a composition provided herein is formulated for topical application to the skin, the composition may be co-formulated with additional active ingredients. Such additional active ingredients may include, for example, antioxidants (e.g., L-ascorbic acid, Niacinamide, Resveratrol, and/or Retinol), hydrating agents (e.g., hyaluronic acid, propylene glycol, alpha hydroxy acids, urea, or glycerin) and/or moisturizing agents (e.g., ceramides and/or dimethicone).

### Methods of Use

A composition provided herein may be used to treat a skin condition, e.g., an inflammatory condition. Thus, provided herein are methods of reducing, treating, preventing, or ameliorating a skin condition, said methods comprising administering a composition described herein to a subject.

As used herein, the term “treating” may refer to, for example, an improvement of one of more symptoms of a condition and/or a delay in disease progression. As used herein, the term “preventing” may refer to, for example, a delay in disease onset compared to, e.g., a population average.

As used herein, the term “subject” may refer to, for example, a patient diagnosed with or suspected of having a skin condition that may benefit from the administration of a composition described herein. The terms “subject” and “patient” are used interchangeably herein. In some embodiments, the subject is human. In some embodiments, the subject is a human adult, e.g., is over 18 years of age, about 18-30 years of age, about 30-40 years of age, about 40-45 years of age, about 45-50 years of age, about 50-55 years of age, about 55-60 years of age, about 60-65 years of age, about 65-70 years of age, about 70-75 years of age, about 75-80 years of age, about 80-85 years of age, about 85-90 years of age, or over 90 years of age.

Inflammatory conditions treated, prevented, reduced, or ameliorated in accordance with the methods described herein may be acute or chronic inflammatory conditions. In some embodiments, the skin condition (e.g., inflammatory condition) treated, prevented, reduced, or ameliorated in accordance with the methods described herein is associated with increased Prostaglandin E2 (PGE₂) expression and/or increased PGE₂ signaling (e.g., PGE₂ signaling via EP1, EP2, EP3 and/or EP4). Non-limited examples of inflammatory skin conditions include dermatitis (e.g., atopic dermatitis) and eczema. In some embodiments, the inflammatory condition treated, prevented, reduced, or ameliorated in accordance with the methods described herein is an adverse drug effect, e.g., a rash.

In some embodiments, the skin condition treated, prevented, reduced, or ameliorated in accordance with the methods described herein is a dermatological symptom of an immunological disorder.

The outcome of a method of treating, preventing, reducing, or ameliorating a skin condition described herein may be determined by studying a biochemical marker of inflammation, for example, PGE₂ expression or expression of inflammatory cytokines (e.g., IL-8 and/or IL-6). In some embodiments, a method of treating, preventing, reducing, or ameliorating a skin condition described herein results in decreased PGE₂ expression and/or decreased PGE₂ signaling. In some embodiments, a method of treating, preventing, reducing, or ameliorating a skin condition described herein results in decreased expression of inflammatory cytokines (e.g., IL-8 and/or IL-6). Expression of PGE₂ and inflammatory cytokines may be determined using any suitable method known in the art or described herein. For example, protein expression may be measured by Enzyme-linked Immunosorbent Assay (ELISA), Western Blotting, or immunofluorescence, while gene expression maybe measured by quantitative real-time PCR or RNA sequencing.

A composition provided herein may be administered using any suitable route of administration. In some embodiments, the composition is administered topically. In some embodiments, the composition is administered orally.

A composition provided herein may be administered at any suitable frequency. In some embodiments, a composition is administered as needed. In some embodiments, a composition is administered daily. In some embodiments, a composition is administered twice a day. In some embodiments, a composition is administered every 2, 3, 4, 5, 6, or 7 days.

### Examples

The invention is further described in the following Examples. The Examples are merely illustrative and do not in any way limit the scope of the invention as described and claimed. This invention can be further illustrated by the following Examples of preferred embodiments thereof, although it will be understood that these Examples are included merely for purposes of illustration and are not intended to limit the scope of the invention unless otherwise specifically indicated.

Abbreviations used herein include the following:

DMEM Dulbecco's modified Eagle's medium

EGF Epidermal growth factor

ELISA Enzyme linked immunosorbent assay

FCS Fetal calf serum

GAM Goat anti-mouse

IL Interleukin

MIC Minimum inhibitory concentration

MW Molecular weight

NHEK Normal human epidermal keratinocytes

NHDF Normal human dermal fibroblasts

OD Optical density

PBS Phosphate buffered saline

PE Pituitary extract

PGE2 Prostaglandin E2

PMA Phorbol myristate acetate

ROS Reactive oxygen species

RT Room temperature

SFM Serum free medium

TGF Transforming growth factor

UV Ultraviolet

λem Emission wavelength

λex Excitation wavelength

Example 1: Effects of six compounds on normal human epidermal keratinocytes The effects on normal human epidermal keratinocytes (NHEK) were measured by assessing IL-8 and PGE₂ release by PMA-stimulated NHEK using specific ELISA kits.

### Materials and Methods

### Biological Models

### Normal Human Epidermal Keratinocytes (NHEK):

| Cell type: | NHEK |
|---|---|
| Culture conditions: | 37° C., 5% CO2 |
| Culture medium: | Keratinocyte SFM supplemented with |
| | Epidermal Growth Factor (EGF) 0.25 ng/ml |
| | Pituitary extract (PE) 25 μg/ml |
| | Gentamycin 25 μg/ml |
| Assay medium 1: | Epilife ® medium supplemented with |
| | PE 0.2% |
| | Bovine transferrin 5 μg/ml |
| | EGF 0.2 ng/ml |
| | Insulin 5 μg/ml |
| | Gentamycin/Amphotericin |
| Assay medium 2: | KeratinocyteSFM supplemented with |
| | Gentamycin 25 μg/ml |

### Test Compounds:

| | | | | | |
|---|---|---|---|---|---|
| CBG | Powder- | 10 mM in | Systemic | NHEK, | 0.3, 1 and |
| Purity: 98% | Storage: | ethanol | | NHDF | 3 μM |
| FD200509-1 | RT | | | | |
| CBD | Powder- | 10 mM in | Systemic | NHEK | 0.1, 0.3 and |
| Purity: 99% | Storage: | ethanol | | | 1 μM |
| FD200509-2 | RT | | | NDHF | 0.3, 1 and |
| | | | | | 3 μM |
| CBGVA | Powder- | 10 mM in | Systemic | NHEK | 0.1, 0.3 and |
| Purity: 97% | Storage: | ethanol | | | 1 μM |
| FD200509-3 | −20° C. | | | NHDF | 1, 3 and |
| | | | | | 10 μM |
| CBDA | Powder- | 10 mM in | Systemic | NHEK | 0.03, 0.1 |
| Purity: 97% | Storage: | ethanol | | | and |
| FD200509-4 | −20° C. | | | | 0.3 μM |
| | | | | NHDF | 1, 3 and |
| | | | | | 10 μM |
| CBGA | Powder- | 10 mM in | Systemic | NHEK | 0.03, 0.1 |
| Purity: 97% | Storage: | ethanol | | | and |
| FD200509-5 | −20° C. | | | | 0.3 μM |
| | | | | NHDF | 1, 3 and |
| | | | | | 10 μM |
| L-Ascorbic | Powder- | 1 mg/ml | Systemic | NHEK, | 50, 100 and |
| acid | Storage: | in culture | | NHDF | 200 μg/ml |
| Ref. Sigma, ref. | RT | medium, | | | |
| A4544 Batch n° | protected | assay | | | |
| SLBX2310 | from light | medium | | | |
| MW: 176.12 | and | or PBS | | | |
| FD200509-6 | humidity | | | | |

### IL-8 and PGE₂ Release by PMA-Stimulated NHEK

### Culture and Treatments

Keratinocytes were seeded in 96-well plates and cultured for 24 hours in culture medium. The medium was then replaced by assay medium 1 containing or not (control) the test compounds or the reference compound (staurosporine tested at 1 nM for IL-8 release or indomethacin tested at 1 μM for PGE₂ release) and the cells were pre-incubated for 24 hours. After pre-incubation, the medium was removed and replaced by assay medium 1 containing or not (stimulated control) the compounds or the reference compound and containing the inducer (PMA tested at 0.5 μg/ml). The cells were then incubated for 24 hours. In parallel, a non-stimulated control condition was performed.

All experimental conditions were performed in n=3. At the end of incubation, the culture supernatants were collected for IL-8 and PGE₂ quantifications.

### Enzyme-Linked Immunosorbent Assay (ELISA)

IL-8 and PGE₂ released in the culture supernatants were measured using specific ELISA kits according to the supplier's instructions.

| | | Low limit of | | High limit of | |
|---|---|---|---|---|---|
| Kit | Supplier | detection | | detection | |
| Human | R&D SystemsRef. DY208 | 31.25 | pg/ml | 2000 | pg/ml |
| IL-8/CXCL8 | Dilution factor: 1/10 | 312.5 | pg/ml | 20 000 | pg/ml |
| DuoSet | | | | | |
| PGE₂ | Enzo Ref. ADI-900-001 | 39.1 | pg/ml | 2 500 | pg/ml |

### Data Management

Raw data were analyzed using Microsoft Excel® software and GraphPad PRISM® software.

The inter-group comparisons were performed by an unpaired Student's t-test. The statistical analysis can be interpreted if n≥5, however for n<5 the statistical values are for information only.

### Results

### Effect on IL-8 Release by PMA-Stimulated Keratinocytes

The effects of compounds A, B, C, D, and E and of L-ascorbic acid on IL-8 released are summarized in Table 1.

TABLE 1
Effect of compounds and L-Ascorbic acid on IL-8
release by PMA-stimulated keratinocytes.

| Treatment | | | | Normalized data |
|---|---|---|---|---|
| Test compound | | Concentration | | % Relative inhibition |
| Non-stimulated | | — | | 100 |
| control* | | | | |
| Stimulated | Control | — | | 0 |
| conditions: | Staurosporine | 1 | μM | 68 |
| PMA- | CBG | 0.3 | μM | 7 |
| 0.5 μg/ml | | 1 | μM | 20 |
| | | 3 | μM | 60 |
| | CBD | 0.1 | μM | 12 |
| | | 0.3 | μM | −44 |
| | | 1 | μM | 18 |
| | CBGVA | 0.1 | μM | 2 |
| | | 0.3 | μM | 11 |
| | | 1 | μM | 9 |
| | CBDA | 0.03 | μM | 11 |
| | | 0.1 | μM | 17 |
| | | 0.3 | μM | 0 |
| | CBGA | 0.03 | μM | 0 |
| | | 0.1 | μM | −7 |
| | | 0.3 | μM | 9 |
| | L-Ascorbic | 50 | μg/ml | −10 |
| | acid | 100 | μg/ml | 11 |
| | | 200 | μg/ml | −15 |

In the non-stimulated control condition, a low basal release of IL-8 was detected in normal human epidermal keratinocytes (NHEK) (80 pg/ml). The treatment of NHEK with PMA at 0.5 μg/ml for 24 hours highly induced IL-8 release (2406 pg/ml) and this effect was significantly inhibited by the reference compound staurosporine tested at 1 nM (68% of relative inhibition). These results were expected and validated the assay.

Under the experimental conditions of this study, CBG, when tested at 3 μM, strongly inhibited the release of IL-8 by PMA-stimulated keratinocytes.

None of the other tested compounds were able to revert PMA stimulation. On the contrary, CBD overstimulated IL-8 release by the keratinocytes when tested at 0.3 μM but this effect was not validated when the compound was tested at other concentrations.

### Effect on PGE₂ Release by PMA-Stimulated Keratinocytes

The effects of compounds and of L-ascorbic acid on PGE2 release are summarized in Table 2.

TABLE 2
Effect of compounds and L-Ascorbic acid on PGE₂
release by PMA-stimulated keratinocytes

| Treatment | | | | Normalized data |
|---|---|---|---|---|
| Test compound | | Concentration | | % Relative inhibition |
| Non-stimulated | | — | | 100 |
| control | | | | |
| Stimulated | Control | — | | 0 |
| conditions: | Indomethacin | 1 | μM | >169 |
| PMA 0.5 | CBG | 0.3 | μM | 86 |
| (μg/ml) | | 1 | μM | >169 |
| | | 3 | μM | >159 |
| | CBD | 0.1 | μM | 0 |
| | | 0.3 | μM | −59 |
| | | 1 | μM | −242 |
| | CBGVA | 0.1 | μM | 19 |
| | | 0.3 | μM | 65 |
| | | 1 | μM | >162 |
| | CBDA | 0.03 | μM | −24 |
| | | 0.1 | μM | −50 |
| | | 0.3 | μM | −20 |
| | CBGA | 0.03 | μM | −17 |
| | | 0.1 | μM | −68 |
| | | 0.3 | μM | −233 |
| | L-Ascorbic | 50 | μg/ml | >148 |
| | acid | 100 | μg/ml | >160 |
| | | 200 | μg/ml | 127 |

In the non-stimulated control condition, a low basal level of PGE₂ was secreted by NHEK (62 pg/ml). The treatment of NHEK with PMA at 0.5 μg/ml for 24 hours resulted in a significant stimulation of PGE₂ release (96 pg/ml) and this effect was completely inhibited by the reference compound indomethacin, tested at 1 μM (>169% of relative inhibition). These results were expected and validated the assay.

CBG, CBGVA, and L-Ascorbic acid strongly and significantly inhibited PGE₂ release by PMA-stimulated keratinocytes (about 160%, 160% and 145% of relative inhibition, respectively). Regarding CBG, the inhibitory effect was significant at all tested concentrations and equivalent when tested at 1 and 3 μM. CBGVA had a significant effect only when tested at the highest concentration. Finally, L-Ascorbic acid induced overall the same effect at all tested concentrations.

On the other hand, CBD and CBGA overstimulated PGE₂ release by PMA-stimulated keratinocytes reaching respectively 185% and 182% of the control when tested at the highest concentration. Finally, CBDA did not modulate PGE₂ release by PMA-stimulated keratinocytes.

### CONCLUSIONS

In the conditions of this study, L-Ascorbic acid significantly limited PGE₂ release from PMA-stimulated keratinocytes

CBG significantly inhibited the release of IL-8 and PGE₂ from PMA-stimulated keratinocytes.

CBGVA inhibited PGE₂ release from PMA-stimulated keratinocytes.

As used herein, all percentages (%) are percent by weight of the total composition, also expressed as weight/weight %, % (w/w), w/w, w/w % or simply %, unless otherwise indicated.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. A skin care product to reduce, treat, prevent, or ameliorate a skin condition, comprising one or more cannabinoid compounds comprising cannabigerol (CBG) and cannabigerovarinic acid (CBGVA).

2. The skin care product of claim 1 comprises about 0.01 μg/mL to about 1 mg/mL of the one or more cannabinoid compounds.

3. The skin care product of claim 1 is formulated for topical application.

4. The skin care product of claim 3 is a lotion, a cream, or an ointment.

5. The skin care product of claim 1 is formulated for oral administration.

6. The skin care product of claim 5 is a pill, an oil, a syrup or a tablet.

7. The skin care product of claim 1, wherein the skin condition is an inflammatory condition.

8. The skin care product of claim 7, wherein the inflammatory condition is an acute inflammatory condition.

9. The skin care product of claim 8, wherein the inflammatory condition is a chronic inflammatory condition.

10. The skin care product of claim 8, wherein the inflammatory condition is associated with increased prostaglandin E2 (PGE2) expression.

11. The skin care product of claim 1, wherein the skin condition is atopic dermatitis.

12. The skin care product of claim 1, wherein the skin condition is a sign or symptom of ageing.

13. The skin care product of claim 12, wherein the sign or symptom of ageing is wrinkling of the skin.

14. The skin care product of claim 1, wherein the skin condition is the dermatological symptom of an immunological disorder.

15. The skin care product of claim 1 reduces PGE2 production in the skin.

16. The skin care product of claim 1 reduces Interleukin 8 (IL-8).

16. The skin care product of claim 1 comprises one or more additional active ingredient.

17. A method of reducing prostaglandin E2 (PGE2) in skin, comprising contacting skin with an effective amount of one or more cannabinoid compounds; and
wherein the one or more cannabinoid compounds comprise cannabigerol (CBG) and cannabigerovarinic acid (CBGVA).

19. The method of claim 18 further reduces Interleukin 8 (IL-8).
